(19) United States
(12) Patent Application Publication
(10) Pub. No.: US 2018/0068759 A1
(43) Pub. Date: Mar. 8, 2018

(54) Stretchable Electrode Conductor Arrangement and Medical Implant

(71) Applicant: BIOTRONIK SE & Co. KG, Berlin (DE)

(72) Inventors: Eckardt Bihler, Winterthur (CH); Marc Hauer, Uster (CH); Daniel Schulze, Bassersdorf (CH)

(21) Appl. No.: 15/664,510

(22) Filed: Jul. 31, 2017

Related U.S. Application Data
(60) Provisional application No. 62/383,604, filed on Sep. 6, 2016.

(51) Int. Cl. H01B 5/14; A61B 5/00; A61N 1/05

(52) U.S. Cl. CPC H01B 5/14; A61N 1/05; A61B 5/686; A61B 5/6853

(57) ABSTRACT

A stretchable electrode conductor arrangement for a medical implant, this stretchable electrode conductor arrangement having at least one zigzag or meandering conductor track on an insulating support with an insulating cover that is tightly connected with the support, embedding the conductor track, the support having an essentially non-stretchable material and being cut in a zigzag or meandering pattern to adapt it to the contour of the conductor track(s).

### CROSS-REFERENCE TO RELATED APPLICATIONS

This patent application claims the benefit of co-pending U.S. Provisional Patent Application No. 62/383,604, filed on Sep. 6, 2016, which is hereby incorporated by reference in its entirety.

### TECHNICAL FIELD

The present invention relates to a stretchable electrode conductor arrangement for a medical implant, this stretchable electrode conductor arrangement having at least one zigzag or meandering conductor track on an insulating support with an insulating cover that is tightly connected with the support, embedding the conductor track. It also relates to a medical implant with such an electrode conductor arrangement.

### BACKGROUND

Electronic medical implants such as, for example, as cardiac pacemakers, implanted cardioverters, cochlear implants, and neurostimulators, exert an effect on excitable body tissues of the patient through electrodes (also referred to as electrode poles), which are in contact with the body tissues. Conversely, electrodes in contact with suitable body tissues can be used to detect endogenous potentials and make them available for diagnostic evaluation.

In most cases, the electrodes or electrode poles are put on long electrode leads or special catheters, but in certain arrangements can also be put on the stimulation device itself or arranged in the form of a planar net or mat. Also known are elastically expandable electrode arrangements, which are advantageous for certain applications. An expandable implant which has an integrated sensor system and such a stretchable electrode arrangement is described in International Publication No. WO 2002/058549.

In the stretchable electrode arrays known up to now, the electrically conductive layers are put on stretchable materials and encapsulated with stretchable materials. The conductor tracks are arranged in a meandering pattern on the stretchable base material. Stretching produces tensile forces between the metal layers and the stretchable base material, which limit the maximum number of stretching cycles and the maximum stretching. The tensile forces can lift the conductor tracks off the base material. The hollow spaces along the conductor tracks can give rise to migration paths for electrolytes, which can cause the structures to fail, or reduce their life.

The present invention is directed toward overcoming one or more of the above-identified problems.

### SUMMARY

Therefore, the present invention has a goal of indicating a stretchable electrode conductor arrangement that is functionally reliable and suitable for long-term operation in the implanted state. Furthermore, a correspondingly reliable medical implant should also be made available.

At least this is accomplished by an electrode conductor arrangement having the features of claim 1 and a medical implant having the features of claim 14. Expedient further developments of the inventive idea are the subject of the dependent claims.

The present invention is to form the support of the electrode arrangement from an essentially non-stretchable material that exerts only small tensile forces on the interface to the conductor tracks when the arrangement is stretched. This can ensure a longer life during operation, even with frequent stretching by relatively large amounts. The present invention also includes realizing the required stretchability of the arrangement as a whole by cutting the support in a zigzag or meandering pattern to adapt it to the contour of the conductor track(s). Thus, the stretchability of the support, like that of the conductor tracks, is realized by the special geometric configuration.

Simply expressed, the inventive arrangement can be imagined as a type of multilayer zigzag or meandering ribbon cable, in particular one which is made from biocompatible materials for the intended use in medical implants. A meandering arrangement is understood to mean various specific configurations of the support, in which the contour can be defined, for example, by alternating cut-out rectangles or trapezoids, or by U-shaped or also Ω-shaped cutouts. The outside contour of the correspondingly shaped support and the cover need not strictly correspond to the conductor track configuration but, rather, can perfectly well deviate from it somewhat, as long as the geometrically determined stretchability of the support (and optionally of the cover) is ensured.

In one embodiment of the present invention, the cover also has an essentially non-stretchable material and is cut in a zigzag or meandering pattern to adapt it to the contour of the conductor track(s). From the current perspective this is the embodiment that is realized in practice, however, in theory, it is also possible for a more strongly stretchable material to be used for the cover than for the support of the arrangement.

Furthermore, in the practical embodiment, the proposed electrode conductor arrangement will normally have at least one electrode pole—typically multiple electrode poles—which is (are) electrically connected with the (at least) one conductor track and which is embedded in the support or the cover and has an exposed surface. Alternatively, the electrode pole(s) can be arranged on the exposed surface of the support or the cover and be connected with the associated embedded conductor track through a via contact.

However, in theory, the proposed electrode arrangement can also be used without integrated electrode poles, in particular by connecting it to separate electrode poles, which can be provided as tip or ring electrodes, for instance at the distal end of an electrode lead.

In other embodiments, the proposed electrode conductor arrangement has multiple parallel conductor tracks and cutouts in the support, and optionally also in the cover that are adapted to the course of the conductor tracks. In particular, the arrangement can be configured as a network of intersecting conductor tracks and have cutouts in the support and optionally also in the cover that are essentially polygonal, e.g., rectangular or parallelogram-shaped. Here it is important for the mentioned cutouts in the support and possibly also in the cover to have zigzag or meandering edges corresponding to the course of the adjacent conductor tracks, to achieve the sought-after stretchability.

In another embodiment, an electrode conductor arrangement of the last-mentioned type has a matrix-shaped array of electrode poles, in particular, electrode poles that are arranged at crossing points or connection points of multiple conductor tracks. Such an arrangement can advantageously realize a stimulation and/or sensing of larger surface sections of a human or animal body.

In the context of this invention, it is essential that the material used for the support be clearly less stretchable than materials used up to now. If this material is qualified as essentially non-stretchable, this does not mean that it may not have any stretchability. Suitable materials for the support and/or the cover are a liquid crystal polymer (LCP), or a polyimide (PI). However, other types/classes of plastics can also be used, depending on the application situation.

In embodiments that are useful from the current perspective, the thickness of the support and/or the cover is in the range between 5 and 100 μm, and the thickness of the, or every, conductor track is in the range between 1 and 20 μm. It goes without saying that the thicker the material layers are, the more the non-stretchability of the support and cover is ensured. However, a greater thickness is inevitably accompanied by a higher bending rigidity and other disadvantages, so that to achieve the above-mentioned advantages and to avoid application disadvantages, the embodiment of the present invention must ultimately find a compromise between the material-specific stretchability and the material thickness.

Furthermore, from the current perspective, the practical embodiment provides that the width of the, or every, conductor track is in the range between 2 and 500 μm, especially between 10 and 100 μm, and the local width of the support is greater than the width of the conductor track and is in the range between 25 and 250 μm. However, in principle, other widths are also conceivable for special applications, if an inventive function is ensured by corresponding selection of the type and thickness of the material.

In other embodiments of the present invention, the composite of the support, conductor track(s), and cover is put on an elastically deformable, especially stretchable planar base or embedded into such a base. This is normally understood to be an additional base (that is, a fourth material layer), however, in principle a functional combination of a (in this case stretchable) cover and base in a single component is also conceivable. The base can have a polyurethane or silicone material or a rubber.

In embodiments of this design, the base is spatially shaped as a cylinder or balloon, or it has a cylindrical or balloon-shaped section. Such embodiments can realize, for example, novel electrode leads or balloon catheter arrangements with a stimulation or sensing function.

In one embodiment of the proposed implant, it comprises an additional functional component inserted into the electrode conductor arrangement, for instance, an electronic control and/or evaluation component. In particular, this component can be placed between the electrode conductor arrangement itself and a stretchable base supporting or enveloping it.

Further features, aspects, objects, advantages, and possible applications of the present invention will become apparent from a study of the exemplary embodiments and examples described below, in combination with the Figures, and the appended claims

### DESCRIPTION OF THE DRAWINGS

Other advantages and expedient features of the present invention follow from the following description of sample embodiments, which make reference to the Figures. The Figures are as follows:

FIGS. 1A and 1B show a schematic sectional view and top view, respectively, of an electrode conductor arrangement according to one embodiment of the present invention;

FIG. 2 shows a schematic top view of an electrode conductor arrangement according to another embodiment of the present invention;

FIG. 3 shows a schematic top view of an electrode conductor arrangement according to another embodiment of the present invention;

FIG. 4 shows a schematic representation of a special use of the electrode conductor arrangement according to FIG. 3;

FIG. 5 shows a schematic representation of a medical implant according to an embodiment of the present invention;

FIGS. 6A and 6B show a perspective representation and a partial view, respectively, of a medical implant according to another embodiment of the present invention; and

FIGS. 7A and 7B show schematic sectional views of a medical implant according to another embodiment of the present invention.

### DETAILED DESCRIPTION

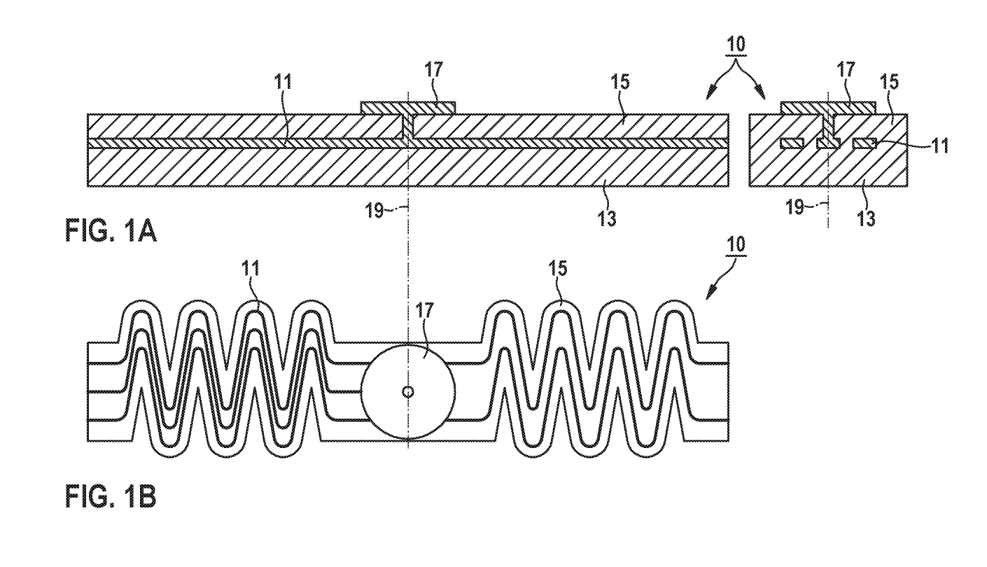

FIGS. 1A and 1B show a three-layer electrode conductor arrangement 10 in which multiple conductor tracks 11 having a parallel zigzag course are embedded between a first non-stretchable polymer layer (support) 13 and a second non-stretchable polymer layer (cover) 15, the first and second polymer layers 13, 15 having an outside contour that is cut to adapt it to the zigzag course of the conductor tracks 11. The cutting can be done, for instance, by means of a laser, or also by milling or plasma etching, or possibly by means of a punch.

In the embodiment shown, a disk-shaped electrode pole 17 laid on the cover 15 and a via 19 are provided in a middle area between conductor tracks on both sides and associated support/covering areas, the via 19 connecting the electrode pole 17 with the conductor tracks 11.

The zigzag arrangement of the conductor tracks 11 and the corresponding zigzag-shaped cut of support 13 and the cover 15 allow the areas of the electrode conductor arrangement 10 bordering the electrode pole 17 on both sides of the middle part to be elastically stretched in the longitudinal direction without any problems, without repeated and even pronounced stretching leading to the above-described disadvantages, such as occur after a longer time in the case of support/covering composites made of intrinsically stretchable materials.

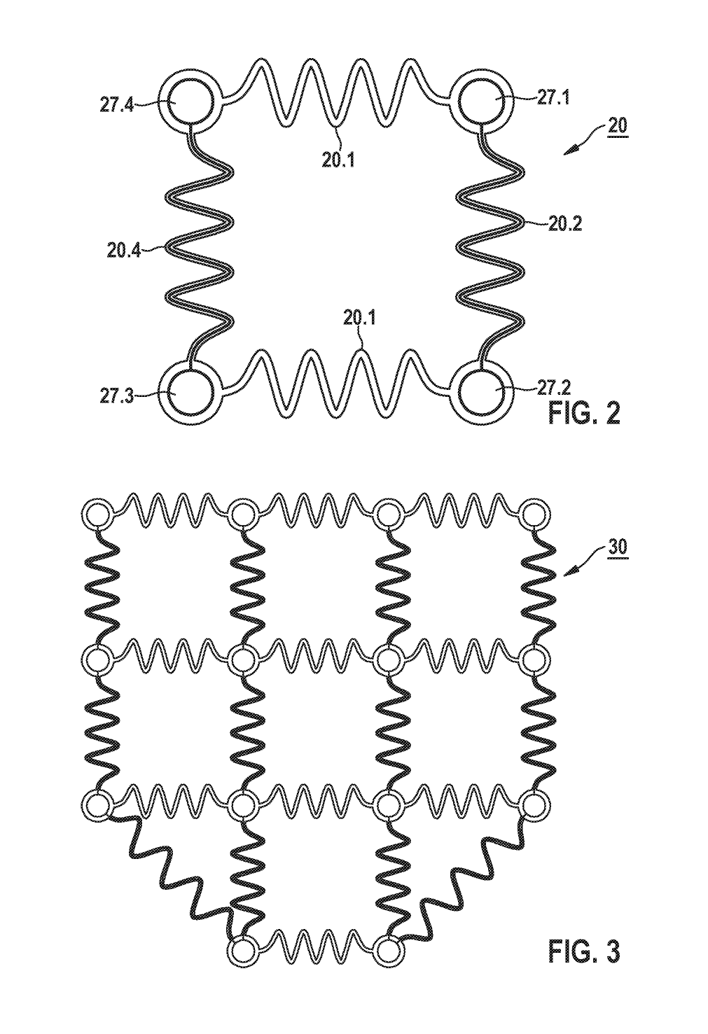

FIG. 2 shows a top view of another example of an electrode conductor arrangement 20, which comprises four support/conductor track/cover strands 20.1-20.4 in the basic shape of a square, and one electrode pole 27.1-27.4 at each of the vertices.

FIG. 3 shows another example of an electrode conductor arrangement 30, which comprises six subarrangements of the type shown in FIG. 2 in a 2×3 matrix arrangement, and in addition one rectangular and two triangular subarrangements on either side of it.

To provide additional mechanical stability to the structure and adapt it better to the tissue, the cut-out meandering structure can be embedded in a stretchable polymer. This polymer can be either in the form of a closed film or be perforated, so that the structure can grow into the body tissues. The embedding can either be done by pressing in at an elevated temperature and pressure, the temperature being raised above the glass-transition temperature of the second polymer (e.g., PU), but still below that of the first polymer (e.g., LCP), or on the other hand by molding in a mold with a second polymer (e.g., silicone resin).

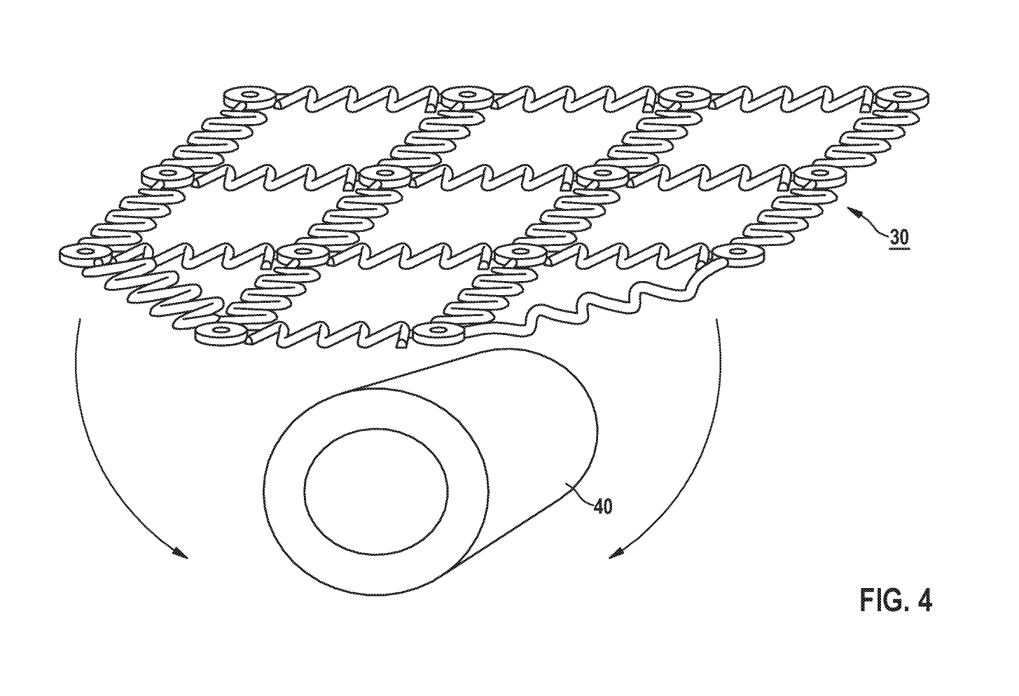

FIG. 4 schematically shows how the arrangement 30, shown in top view in FIG. 3, is applied to the periphery of a tubular base 40. This makes it possible, for example, to equip the periphery of a catheter with a regular array of electrode poles, in order, for example, to stimulate a longer section of a vessel, or to sense tissue potentials there. The electrode conductor arrangement 30 can be applied by pressing it on at elevated temperature, or, if a silicone tube is used as a base 40, it can also be done by inserting the electrode conductor arrangement into a corresponding mold and filling it with the silicone material.

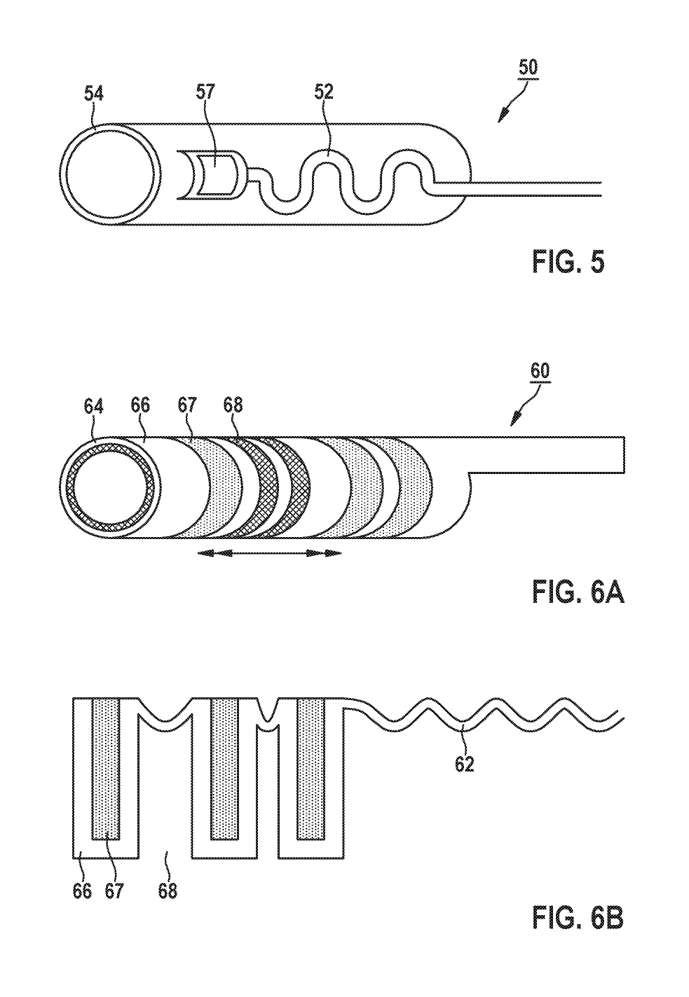

FIG. 5 schematically shows, as another example of a use of the inventive electrode conductor arrangement, a distal end section of an electrode lead 50 that is equipped with an essentially rectangular electrode pole 57. A meandering lead 52, which is realized by a support/conductor track/cover composite of the above-described type, is applied to a stretchable silicone or PU tube 54 as a base body.

A schematic perspective representation or detail view of a modified embodiment of an electrically active catheter 60 is shown in FIGS. 6A and 6B. Here, the base body is once again a stretchable PU or silicone tube 64, onto which each of three ring electrodes 67 is applied onto a substrate 66 in such a way that expansion joints 68 remain between them. The ring electrodes 67 are connected through an inventive lead structure 62, which once again has a zigzag or meandering course.

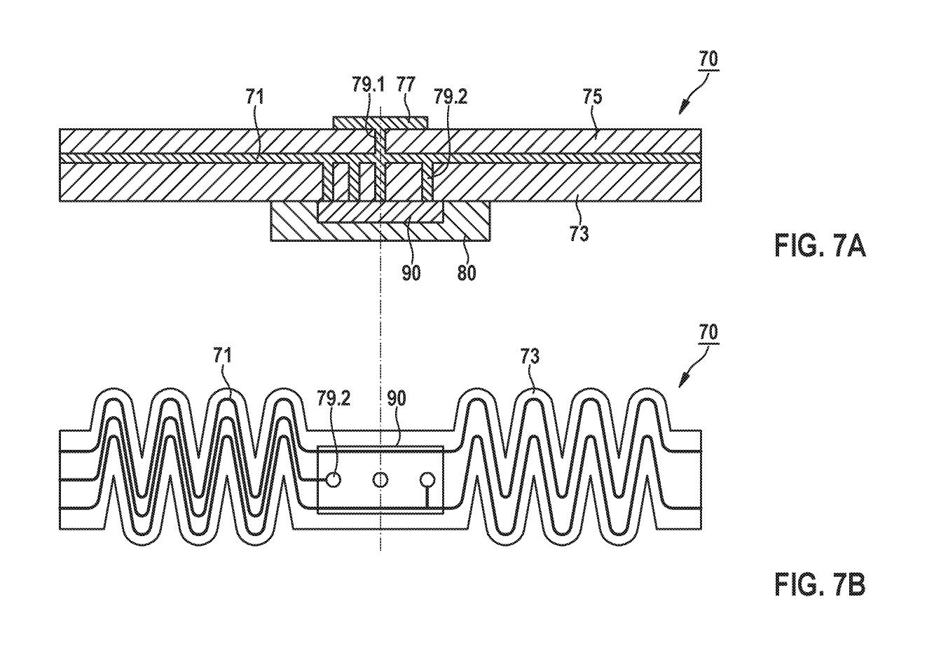

FIGS. 7A and 7B are schematic sectional views showing perpendicular cutting planes through another medical implant 70, whose structure is, in theory, very similar to that shown in FIGS. 1A and 1B and to the electrode conductor arrangement 10 described further above. Therefore, the reference numbers for the individual parts are based on those in FIGS. 1A-1B. The description relating to the parts is not repeated here.

In addition, the arrangement comprises a third polymer layer 80, which is arranged beneath a section of the support 73 and which has a functional component 90 embedded in it. The component 90 is connected to the conductor tracks 71 of the arrangement through second vias 79.2, which extend from the conductor tracks 71 in the opposite direction relative to the first vias 79.1 of the electrode pole 77. The additional component 90 can be, for example, sensors or ultrasonic transducers, which should be connected through the conductor tracks 71 of the electrode conductor arrangement for signal impingement or derivation.

Many other variants of the embodiments of the present invention shown here in the examples and aspects of the invention emphasized further above are possible.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

I/We claim:

1. A stretchable electrode conductor arrangement for a medical implant, the stretchable electrode conductor arrangement comprising:
at least one zigzag or meandering conductor track on an insulating support with an insulating cover that is tightly connected with the support, embedding the conductor track,
the support having an essentially non-stretchable material and being cut in a zigzag or meandering pattern to adapt it to the contour of the conductor track(s), making it stretchable as a whole.

2. The electrode conductor arrangement according to claim 1, wherein the cover also has an essentially non-stretchable material and is cut in a zigzag or meandering pattern to adapt it to the contour of the conductor track(s).

3. The electrode conductor arrangement according to claim 1, which has at least one electrode pole which is electrically connected with the at least one conductor track and which is embedded in the support or the cover and has an exposed surface.

4. The electrode conductor arrangement according to claim 1, which has at least one electrode pole which is electrically connected with the at least one conductor track and which is arranged on the exposed surface of the support or the cover and is connected with the embedded conductor track through a via contact.

5. The electrode conductor arrangement according to claim 1, which has multiple parallel conductor tracks and cutouts in the support, and optionally also in the cover, that are adapted to the course of the conductor tracks.

6. The electrode conductor arrangement according to claim 5, which has a network-like arrangement of intersecting conductor tracks and cutouts in the support and optionally also in the cover that are essentially polygonal.

7. The electrode conductor arrangement according to claim 6, with a matrix-shaped array of electrode poles, in particular electrode poles that are arranged at crossing points or connection points of multiple conductor tracks.

8. The electrode conductor arrangement according to claim 1, wherein the support and/or the cover has a liquid crystal polymer (LCP), or a polyimide (PI).

9. The electrode conductor arrangement according to claim 1, wherein the thickness of the support and/or the cover is in the range between 5 and 100 μm, and the thickness of the or every conductor track is in the range between 1 and 20 μm.

10. The electrode conductor arrangement according to claim 1, wherein the width of the, or every, conductor track is in the range between 2 and 500 μm, and the local width of the support is greater than the width of the conductor track and is in the range between 25 and 250 μm.

11. The electrode conductor arrangement according to claim 1, wherein the composite of the support, conductor track(s), and cover is put on an elastically deformable, especially stretchable planar base or embedded into such a base.

12. The electrode conductor arrangement according to claim 11, wherein the base has a polyurethane or silicone material or a rubber.

13. The electrode conductor arrangement according to claim 11, wherein the base is spatially shaped as a cylinder or balloon, or it has a cylindrical or balloon-shaped section.

14. A medical implant, especially an electrode lead, electrode mat, or balloon catheter, with an electrode conductor arrangement according to claim 1.

15. A medical implant according to claim 14, wherein the electrode conductor arrangement is associated with an additional functional component, especially an electronic control and/or evaluation component, and this component is placed especially between the electrode conductor arrangement and an associated base.
